(19) United States
(12) Patent Application Publication
(10) Pub. No.: US 2023/0302182 A1
(43) Pub. Date: Sep. 28, 2023

(54) SYSTEM AND METHOD FOR DISINFECTING VEHICULAR SURFACES

(71) Applicant: International Automotive Components Group GmbH, Düsseldorf (DE)

(72) Inventors: Sung-Uk KIM, Grafing (DE); Carter Scott CANNON, Munich (DE)

(21) Appl. No.: 18/040,885

(22) PCT Filed: Aug. 9, 2021
(86) PCT No.: PCT/IB2021/057338

§ 371 Date: Feb. 7, 2023

Related U.S. Application Data
(60) Provisional application No. 63/063,699, filed on Aug. 10, 2020.

(51) Int. Cl. A61L 2/24; A61L 2/10; B60H 1/00; B60H 3/00

(52) U.S. Cl. CPC A61L 2/24; A61L 2/10; B60H 1/00742; B60H 3/0071; A61L 2202/11; A61L 2202/14; A61L 2202/16; A61L 2202/25

(57) ABSTRACT

A system for disinfecting surfaces in a space, such as a vehicle or a building. The system may include a disinfecting module having a UVC light source and/or an ambient heat source for disinfecting the space. In some embodiments, an occupancy sensor is incorporated within the module. In some embodiments, control of the system in incorporated into the functionality of a ride-share application to facilitate disinfection of the vehicle between occupant rides.

### RELATED APPLICATIONS

This disclosure is a claims the benefit of U.S. Provisional Application Ser. No. 63/063,699 entitled “System and Method for Disinfecting Vehicular Surfaces” filed on Aug. 10, 2020, which is incorporated by reference herein in its entirety.

### TECHNICAL FIELD

The present application relates to generally to systems and methods for disinfecting vehicular surfaces.

### BACKGROUND

Disinfection of surfaces in spaces occupied by people or animals is of interest in a wide variety of applications for the purpose of preventing exposure to, and spread of bacteria, germs and infectious diseases. Methods of disinfecting surfaces include illuminating the surfaces with ultraviolet light in the c-band of wavelengths between about 200 nm and 280 nm (hereinafter UVC) or exposing the surfaces to relatively high heat

Often, disinfection of spaces such as a vehicle interior, the interior of a home, an office space, etc., is not practical when the space is occupied by a person or animal. Also, placement of UVC lighting or heating elements in such spaces can be a challenge given limited area available for packaging, mounting, the desire to illuminate specific target surfaces and the desire to provide functional and/or ambient lighting in the space. Known systems for disinfecting surfaces using UVC and/or heat have not provided a simple and efficient way of confirming to occupants that disinfection is now in process or has been completed prior to their entry into the space.

### BRIEF DESCRIPTION OF THE DRAWINGS

The above-mentioned and other features of this disclosure, and the manner of attaining them, will become more apparent and better understood by reference to the following description of embodiments described herein taken in conjunction with the accompanying drawings, wherein:

FIG. 1 is a simplified block diagram of one example of a system consistent with the present disclosure.

FIG. 2 is top plan view of one example of a lighting module consistent with the present disclosure.

FIG. 3 is top plan view of another example of a lighting module consistent with the present disclosure (with Occupancy Sensor).

FIG. 4 is a cross-sectional view of an example embodiment of an interior trim assembly including a lighting system consistent with the present disclosure.

FIG. 5 is an exploded view of the embodiment shown in FIG. 4.

FIG. 6 is a cross-sectional view of another example embodiment of an interior trim assembly including a lighting system consistent with the present disclosure.

FIG. 7 is an exploded view of the embodiment shown in FIG. 6.

FIG. 8 is a cross-sectional view of another example embodiment of an interior trim assembly including a lighting system consistent with the present disclosure.

FIGS. 9A-9C illustrate successive steps in assembling the embodiment shown in FIG. 8.

FIG. 10 is a cross-sectional view of another example embodiment of an interior trim assembly including a lighting system consistent with the present disclosure.

FIGS. 11A-11B illustrate successive steps in assembling the embodiment shown in FIG. 10.

FIG. 12A is a side sectional diagrammatic view of a system consistent with the present disclosure positioned in a door trim assembly illuminating portions of a vehicle door with ambient light.

FIG. 12B is a front diagrammatic view of the system shown in FIG. 12A showing portions of the door illuminated by the ambient light.

FIG. 13A is a side sectional diagrammatic view of the system shown in FIG. 12A illuminating portions of a vehicle door with UVC light.

FIG. 13B is a front diagrammatic view of the system shown in FIG. 12A showing portions of the door illuminated by the UVC light.

FIG. 14 is a simplified block diagram of another example of a system consistent with the present disclosure.

FIG. 15 illustrates a cross-section of a trim panel showing the use of a plurality of heating elements providing the indicated heat flow.

FIG. 15A illustrates a cross-section of a trim panel where the heating element are positioned between the cover layer and backing material.

FIG. 15B illustrates a cross-section of a trim panel where the heating elements are laminated to the substrate.

FIG. 15C illustrates a cross-section of a trim panel where the heating elements are placed within the substrate.

FIG. 15D illustrates a cross-section of a trim panel where the heating elements are within the substrate and where there is no backing material of cover layer.

FIG. 15E illustrates a cross-section of a trim panel where the heating elements are on the surface of the substrate.

FIG. 15F illustrates a cross-section of a trim panel where the heating elements are within the backing layer which is beneath the cover layer.

FIG. 16 illustrates a typical vehicle that may be configured to include the heating disinfecting modules and/or UVC disinfecting system.

FIG. 16A illustrates the surfaces (shaded) that me be selectively heated and/or selectively exposed to UVC for disinfection.

### DETAILED DESCRIPTION

The present disclosure is not limited in its application to the details of construction and the arrangement of components set forth in the following description or illustrated in the drawings. The examples described herein may be capable of other embodiments and of being practiced or being carried out in various ways. Also, it may be appreciated that the phraseology and terminology used herein is for the purpose of description and should not be regarded as limiting as such may be understood by one of skill in the art. Throughout the present description, like reference characters may indicate like structure throughout the several views, and such structure need not be separately discussed. Furthermore, any particular feature(s) of a particular exemplary embodiment may be equally applied to any other exemplary embodiment(s) of this specification as suitable. In other words, features between the various exemplary embodiments described herein are interchangeable, and not exclusive.

In general, systems and methods consistent with the present disclosure preferably include one or more disinfection modules configured for disinfecting interior vehicular surfaces. The disinfection modules may be configured to disinfect surfaces using any known mechanism or combination of mechanisms, e.g. UVC lighting, ambient heat, chemical and vapor phase disinfectants such as hydrogen peroxide, etc. It is contemplated that the disinfection is configured so that the vehicular surfaces at issue, that may be repeatedly disinfected, remain compliant with OEM aging requirements regarding physical properties and/or appearance.

Some embodiments of a lighting system consistent with the present include lighting modules with a combination of functional and/or ambient and disinfecting UVC lighting. In some embodiments, the lighting modules may include only the UVC lighting without the functional or ambient lighting. The system may be configured to provide desired ambient lighting to the space either simultaneously with, or separately from, providing UVC disinfecting lighting to surfaces within the space. In some embodiments, one or more occupancy sensors may be provided to detect whether the space is occupied by a person or animal. The controller may be provided in the lighting module with the ambient and/or UVC lighting. In some embodiments, the controller may be coupled to one or more user devices, e.g. user input/output (i/o) controls (IoT—Internet of Things), a portable electronic device such as a smart phone, smart watch, a remote electronic device, to allow a user to enable the UVC lighting during desired periods of time. In some embodiments, the user device (e.g. external vehicle key-FOB) may be coupled, e.g. through a wireless connection to one or more occupant devices to notify future occupants of the space that the space has been disinfected by illumination with the UVC lighting. The user devices and/or the occupant devices may include an application thereon for controlling the system. In some embodiments, the application may be a ride-sharing or car-sharing Subscription and Service APP application configured/mapped for managing a ride-sharing service using the vehicle in combination with disinfection of surfaces in the vehicle. A future occupant may be alerted via a pop-up notification on the occupant device of the status of the scheduled ride along with status of disinfection of the vehicle.

For simplicity and ease of explanation, embodiments of a system and method consistent with the present disclosure will be described herein in connection with a passenger vehicle, e.g. automobile, Robo-Taxi, SAV, Train, airplane, bus, boat, etc., application. It is to be understood, however, that embodiments consistent with the present disclosure may be implemented in any interior or exterior space that may be occupied by people or animals.

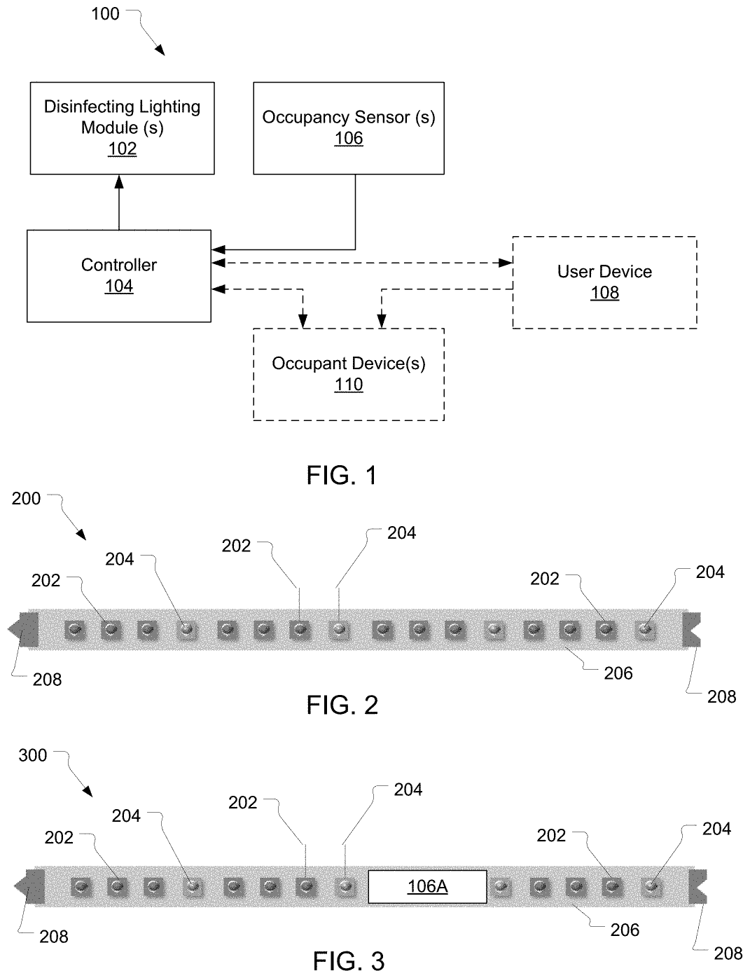

Turning now to FIG. 1, there is illustrated a block diagram of an example embodiment 100 of a system consistent with the present disclosure. The illustrated example embodiment, includes one or more disinfecting lighting modules (s) 102 that is integral or which can interface with the vehicle's central ECU and on-board computer within a vehicle interior. A controller 104 is provided that is configured to control operation of the lighting modules 102 to emit functional and/or ambient and/or UVC light from the lighting modules 102 and one or more occupancy sensors 106. The system may also include an optional user device (with APP) 108 for providing user input to the controller 104 to activate and/or deactivate the lighting modules 102. The system may also or alternatively include one or more occupant devices 110 used and/or viewed by a present or future occupant of the vehicle interior, e.g. passengers. The occupant devices may be coupled interfaced to the user device 108 and/or the controller 104 for receiving confirmation, e.g. a visual or audible confirmation, that the vehicle interior is in process or has been disinfected by UVC light from the lighting modules 102 prior to entry of the occupant into the vehicle interior.

Each of the lighting modules 102 may be provided in a single package including one or more functional and/or ambient light emitting diodes (LEDs) combined with one or more UVC Diodes, e.g. coupled to a common substrate, module or strip. Alternatively, the functional and/or ambient light LEDs may be omitted and only the UVC diodes may be provided in the lighting module 102 without the functional and/or ambient light LEDs. As used herein, the terms, “light emitting diode” and “LED” are used interchangeably and refer to any light emitting diode or other type of carrier injection/junction-based system that is capable of generating radiation in response to an electrical signal, e.g. from the controller 104. Thus, the term LED includes but is not limited to various semiconductor-based structures that emit light in response to current, light emitting polymers, light emitting stripes, electro-luminescent strips, and the like.

In particular, the term LED refers to light emitting diodes of all types (including semi-conductor and organic light emitting diodes), and which may be configured to generate light in all or various portions of one or more of the visible, ultraviolet, and UV spectrum. Non-limiting examples of suitable LEDS that may be used include various types of RGB LEDs, addressable RGB LEDs, single color LEDs, infrared LEDS, ultraviolet LEDS, red LEDS, green LEDS, blue LEDS, yellow LEDS, amber LEDS, orange LEDS, and white LEDS. Such LEDS may be configured to emit light over a broad spectrum (e.g., the entire visible light spectrum) or a narrow spectrum.

The LED used in the present disclosure may be formed by one or a plurality of individual LEDS. For example, the LED light source may be configured to include a number of individual LEDS that emit different spectra but which, collectively, emit light that is of a desired color (e.g., white, red, blue, green, yellow, orange, amber, etc.) and/or color temperature (kelvin—symbol shown as K. An LED may also be associated with one or more phosphors that are an integral part of the LED.

As used herein, the term “color” is used interchangeably with the term “spectrum.” However, the term, “color” generally is used to refer to a property of radiation that is perceivable by an observer (though this usage is not intended to limit the scope of this term). Accordingly, the term “different colors” implies two different spectra with different wavelength components and/or bandwidths. In addition, “color” may be used to refer to white and non-white light.

In some embodiments, one or more of the lighting modules 102 may include at least one red (R), green (G) and blue (B) LED, and optionally at least one yellow (Y) LED. The R, G, B, and optionally Y LEDS each emit light in individual regions of the visible spectrum but, collectively, enable the artificial light sources to emit light of any color, including any or a subset of colors in the RGB and/or RGBY gamut. Alternatively, or additionally, the lighting systems of the present disclosure may make use of color tunable LEDS, i.e., individual LEDs with adjustable color temperature and optionally adjustable intensity. As a non-limiting example of such color tunable LEDS, mention is made of phosphor converting LEDS.

As is known, use of a specific color to describe an LED or the light emitted by the LED refers to a specific range of dominant wavelengths associated with the specific color. In particular, the term “red” when used to describe an LED or the light emitted by the LED means the LED emits light with a dominant wavelength between 610 nm and 750 nm and the term “amber” refers to red light with a dominant wavelength more specifically between 610 nm and 630 nm. The term “green” when used to describe an LED or the light emitted by the LED means the LED emits light with a dominant wavelength between 495 nm and 570 nm. The term “blue” when used to describe an LED or the light emitted by the LED means the LED emits light with a dominant wavelength between 430 nm and 490 nm. The term “yellow” when used to describe an LED or the light emitted by the LED means the LED emits light with a dominant wavelength between 570 nm and 600 nm. The term “white” generally refers to white light with a correlated color temperature (CCT) between about 2600 and 8000 K, “cool white” refers to light with a CCT substantially above 3600K, which is more bluish in color, and “warm white” refers to white light with a CCT of between about 2600 K and 3600 K, which is more reddish in color. The term “UVC” when used to describe an LED or the light emitted by the LED means the LED emits light with a dominant wavelength between 200 nm and 280 nm. UVC light is particularly effective in disinfecting surfaces from viruses.

The LEDs within each lighting module 102 may be independently addressable by the controller 104 for causing emission of light therefrom collectively and/or independently. The controller 104 may include a processor and a memory for storing non-transient computer readable instructions. In operation, the controller 104 outputs control signals to the lighting modules 102, e.g. in response to instructions/program/algorithm stored in memory of the controller 104 and/or input from the user devices 108. Based on the content of the control signals, the functional and/or ambient LEDS of the lighting modules 102 may emit light of a desired color, color temperature and, optionally, intensity and/or the UVC LEDS of the lighting modules 102 may emit UVC light that is imparted on surfaces within the interior of the vehicle to disinfect the surfaces and deactivate any viruses thereon.

The occupancy sensor(s) 106 may be provided at one or more locations in the vehicle interior to detect the presence of an occupant, e.g. a person or animal, in the vehicle. In some embodiments, the occupancy sensors 106 may include known weight sensors in the passenger seats and/or floor, infrared sensors, cameras (object recognition), ultrasonic, microphones, and/or various proximity sensors that may be configured to detect weight, temperature, motion, sound, etc. in the interior of the vehicle. The outputs of the occupancy sensor(s) 106 may be provided to the controller 104 and the controller 104 may be configured to provide control signals to the lighting modules 102 to cause UVC emission from the UVC LEDS only when the occupancy sensor(s) 106 indicate there is no occupant in the vehicle or in the optional portion of the vehicle to be disinfected and/or when the vehicle is secure, e.g. the windows are up, the vehicle is in a park condition, a partition between a front of the passenger compartment and a rear of the passenger compartment is up, etc.

In some embodiments, the occupancy sensor (s) 106 may be incorporated into the lighting modules 102, e.g. in a common strip or substrate with the ambient and UVC LEDS and/or in the same package with the ambient and UVC LEDS. In some embodiments, for example, the occupancy sensors 106 may include one or more known passive or active optical detectors provided on the same substrate or strip with the ambient and/or UVC LEDS.

A passive optical detector may detect ambient light reflected from surfaces within the vehicle and provide a first output when directed at an unoccupied occupant location and a second output when directed at an occupant location that is occupied by person or animal. An active optical detector may include a transmitting portion configured to transmit light toward an anticipated occupant location and a receiver configured to detect portions of the transmitted light reflected from surfaces in the anticipated occupant location. The receiver may provide a first output when the anticipated occupant location is not occupied and a second output when the anticipated occupant location is occupied by person or animal. Incorporating the occupancy sensor(s) 106 in the lighting modules 102 advantageously provides a modular configuration allowing facile retrofit of existing vehicles with a lighting system consistent with the present disclosure and facile localization of the UVC disinfecting light within the interior of the vehicle.

The optional user device(s) 108 are coupled to the controller 104 through a wired or wireless connection and may be cloud-based for providing a user control signal to the controller 104 for selectively activating or programming the functional and/or ambient and/or UVC LEDS in the lighting modules 102. The user device(s) 108 may be any traditional user i/o interface, e.g. mechanical and/or electronic buttons or actuation points on a panel or display or external vehicle key-FOB. In some embodiments, the user device(s) 108 may be wireless (e.g. radio-based or optical) such as a personal computer, smart phone, tablet or other mobile device. The controller 104 may include a transceiver for sending data to/from the wireless user device(s)108. In some embodiments, the user devices(s) 108 may communicate with the controller 104 using a digital communications protocol, such as a digital multiplexer (DMX) interface, a Wi-Fi™ protocol, a digital addressable lighting interface (DALI) protocol, a ZigBee protocol, or any other suitable communications protocol, wired and/or wireless.

Advantageously, wireless user device(s) 108, e.g. smart phones containing applications, may alternatively or additionally be equipped with an application that allows a user to selectively cause emission of light from the UVC LED(s) lighting module(s) 102 for disinfecting the interior of the vehicle, e.g. according to a predetermined schedule and pre-programmed, at times when the occupancy sensors detect there is no occupants in the vehicle and/or at a present time or selected time in the future.

In some embodiments, the wireless user device(s) 108 may include a ride-share application whereby a driver of the vehicle provides transportation to occupants for a subscription/service fee using a ride-sharing/ride hailing and taxi application, such as UBER®, LYFT®, DiDI, MOIA, MyTaxi and car sharing applications such as DriveNow, Miles, and Bolt. The wireless user device may be supplied by vehicle rental companies such as National, SIXT, or AVIS.

Control of a lighting system consistent with the present disclosure may be integrated with the ride-sharing application on the user device 108, whereby management of both the ride-sharing service and disinfection of the vehicle are controlled by the ride-sharing application. The ride-sharing application on the user device may be configured to provide an output to the controller 104 to selectively or automatically cause emission of light from the UVC LED(s) lighting module(s) 102 for disinfecting the interior of the vehicle after one or more occupants leave the vehicle and before additional occupants enter the vehicle, e.g. between rides provided by the user according to the ride-share, car-share, taxi APP, or rental car application. For example, when the ride-share application indicates that an occupant ride has been completed the application may initiate a disinfection of the vehicle through communication with the controller 104 of a lighting system consistent with the present disclosure. Disinfection may be accomplished by imparting UVC light from the lighting modules 102 preferably for a time period in a range from about 1 minute to 30 minutes in some embodiments. Scheduling of occupant rides within the ride-share application may be performed by taking into account the time required for disinfection.

In some embodiments, the ride-share application may provide an alert - notification (pop up on screen) to the user device 108 to alert the user that a disinfection process is about to begin and instruct the user to leave the vehicle during disinfection. In other embodiments, the user may remain in the vehicle and the lighting modules may be localized and controlled by the ride-share application to impart UVC light only to portions of the vehicle, e.g. the back seat or rear of the passenger compartment, where occupants where previously located or are anticipated to be located. Integration of a system consistent with the present disclosure within a ride-share application allows facile disinfection of the occupant spaces between occupant rides and, in some embodiments, may allow a user to control the disinfection and monitor the status of the disinfection within the ride-share application at the user device 108.

In some embodiments, occupant device (s) 110 may alert the occupant(s) that a disinfection process has been completed in the vehicle. The occupant device(s) 110 may include indicator lights and/or known photoluminescent materials incorporated into the disinfected surfaces that luminesce when UVC light is imparted thereon. In some embodiments, the occupant device(s) may be wireless devices (e.g. radio-based or optical) such as a personal computer, smart phone, tablet or other mobile device. The controller 104 may include a transceiver for sending data to/from the occupant device(s) 110. In some embodiments, the occupant device(s) 110 may communicate with the controller 104 using a digital communications protocol, such as a digital multiplexer (DMX) interface, a Wi-Fi™ protocol, a digital addressable lighting interface (DALI) protocol, a ZigBee protocol, or any other suitable communications protocol, wired and/or wireless. The controller 104 may wirelessly provide a signal to the occupant device 110 to provide an indication, e.g. visual or audible, that disinfection of the interior of the vehicle has been completed.

Advantageously, wireless occupant device(s) 110, e.g. smart phones, may additionally or alternatively be equipped and/or mapped to the ride-sharing application and the controller 104 and/or the user devices(s) 108 may provide an output to the ride-sharing application on the occupant device(s) 110 indicating that disinfection of the vehicle has been completed before the occupant with the occupant device 110 enters the vehicle. In addition, the occupant devices (11) may simultaneously communicate with a vehicle feel owner or vehicle driver with recorded history regarding vehicle disinfection. The ride-sharing application may thus provide an indication to a future occupant of the vehicle of the vehicle location and status of disinfection. For example, the ride sharing may provide a message (e.g. pop-up on screen) to the future occupant on the occupant device 110 such as: “Your ride is 5 minutes away and has been disinfected” or “Your ride is 5 minutes away and disinfection will be complete in 2 minutes.” Advantageously, the ride-share application may thus be integrated with a lighting system consistent with the present disclosure to allow disinfection of the occupant spaces within the vehicle between occupant rides and alerting of the occupants that the vehicle has been disinfected.

The functional and/or ambient and/or UVC LEDs, and optionally the occupancy sensors 106, may be incorporated into a lighting module 102 consistent with the present disclosure in a variety of ways. FIG. 2 illustrates one example embodiment 200 of a lighting module 102 including ambient LEDS 202 and UVC LEDS 204 on a common substrate 206. In the illustrated embodiment, the ambient LEDs 202 and UVC LEDS 204 may be separately addressable by the controller 104 to allow selective control of the lighting module 102 to provide ambient and/or UVC lighting. The controller 104 may be coupled to one or more electrodes or connectors 208 coupled to the strip. Again, the lighting module 102 need not include the ambient LEDS.

FIG. 3 illustrates an example 300 of an lighting module 102 consistent with the present disclosure including ambient LEDS 202, UVC LEDS 204 and an occupancy sensor 106A on a common substrate 206. As discussed herein, the occupancy sensor 106A may be a passive or active optical occupancy sensor. Incorporating the occupancy sensor 106A on the common substrate 206 or strip with the ambient 202 and/or UVC LEDs 204 provides a modular system that facilitates retro-fit of existing spaces and localization of the light output and occupancy sensing provided by a system consistent with the present disclosure.

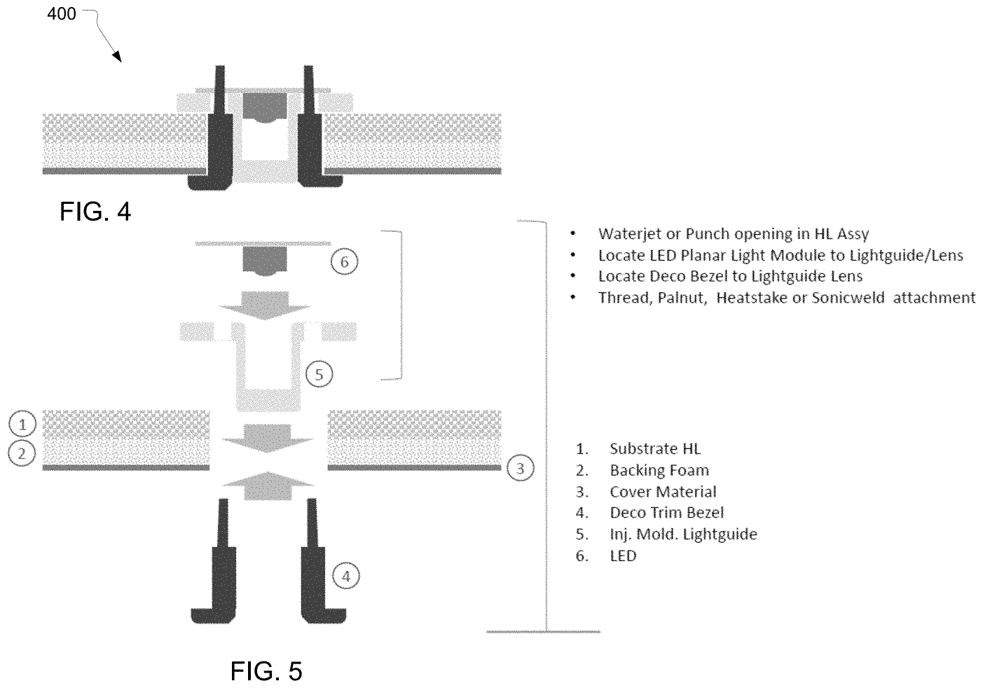

A lighting system consistent with the present disclosure may be incorporated into an interior vehicle trim component, headliner trim, door trim, decorative trim, etc. or overhead lighting, localized lighting, map reading lighting, overhead console etc., in a variety of configurations. FIG. 4 is a cross-sectional view of one embodiment 400 of an interior trim assembly including a lighting system consistent with the present disclosure and FIG. 5 is an exploded view of the embodiment 400 shown in FIG. 4. The illustrated example embodiment includes a substrate, a backing foam, cover material, a decorative (Deco) trim bezel and an injection molded light guide. The ambient and or UVC LEDS on the substrate 106 are positioned in the light guide as shown to emit light through an opening in the substrate, foam and cover material and through the light guide. The bezel covers the edges of the opening to provide a finished appearance.

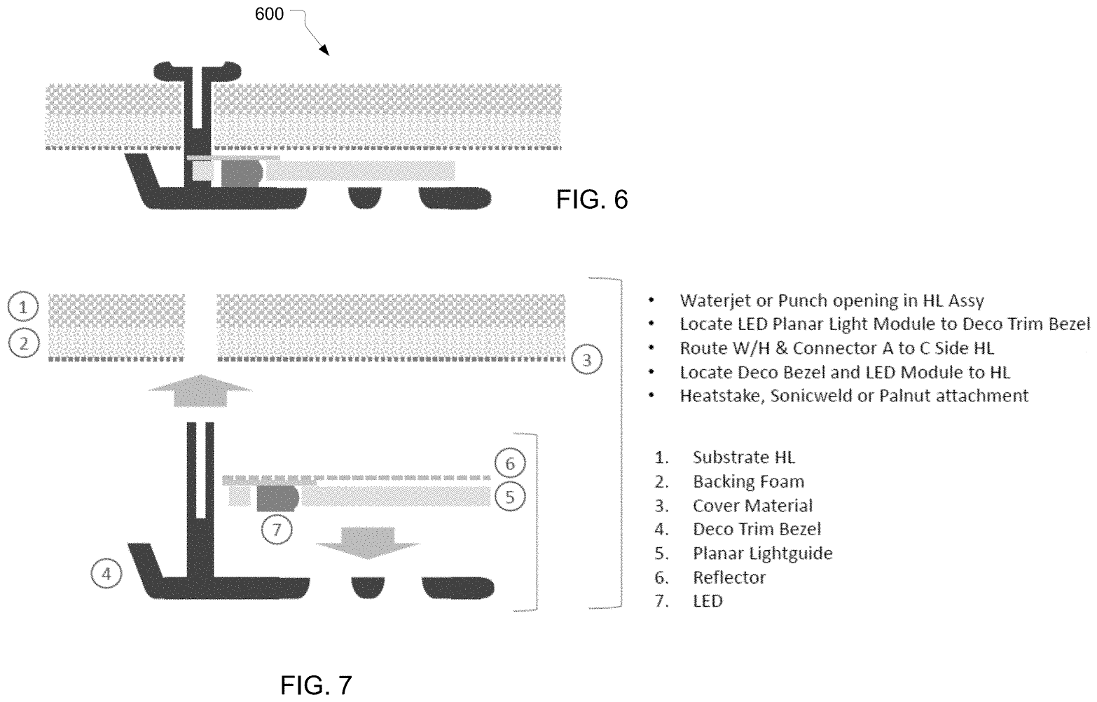

FIG. 6 is a cross-sectional view of another embodiment 600 of an interior trim assembly including a lighting system consistent with the present disclosure and FIG. 7 is an exploded view of the embodiment 600 shown in FIG. 6. The illustrated example embodiment includes a substrate, a backing foam, cover material, a decorative (Deco) trim bezel and a planar light guide. The ambient and or UVC LEDS on the substrate 106 are positioned relative light guide as shown to emit light through a bottom surface of the lightguide in the direction illustrated by the arrow. Light emitted from the LEDS in a direction toward the reflector is reflected from the reflector an emitted from a bottom surface of the lightguide in the direction illustrated by the arrow. The assembly is secured in an opening in the substrate, foam and cover material and the covers the edges of the opening to provide a finished appearance.

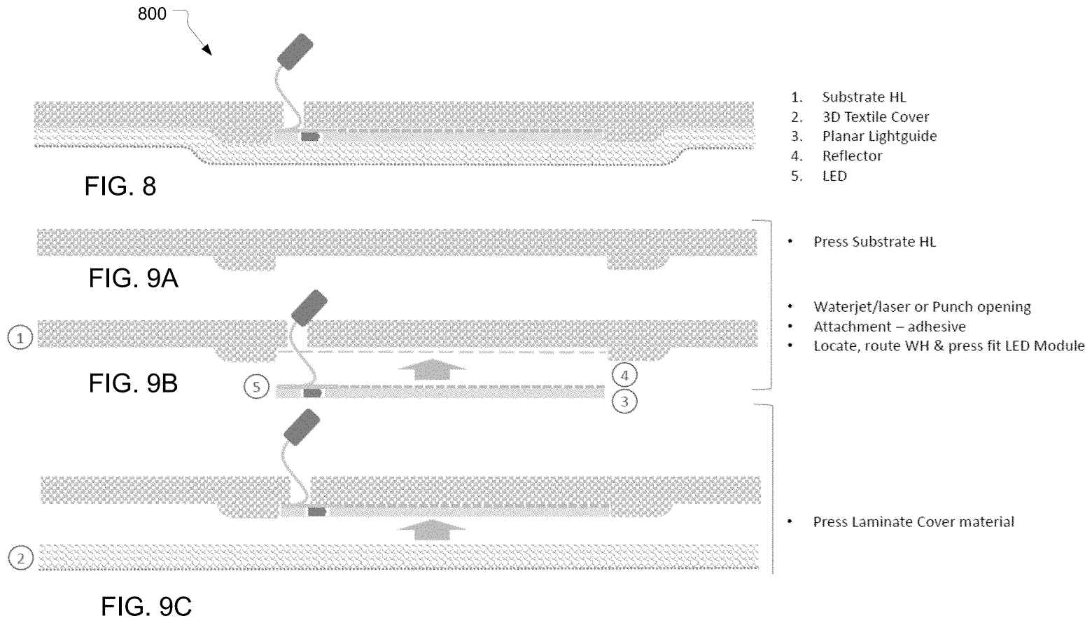

FIG. 8 is a cross-sectional view of another embodiment 800 of an interior trim assembly including a lighting system consistent with the present disclosure and FIGS. 9A-9C illustrate successive steps in assembling the embodiment 800 shown in FIG. 8. The illustrated example embodiment includes a substrate, a 3D textile cover and a planar light guide. The ambient and or UVC LEDS on the substrate 106 are positioned between projections formed in the substrate and relative light guide as shown to emit light through a bottom surface of the lightguide. Light emitted from the LEDS in a direction toward the reflector is reflected from the reflector an emitted from a bottom surface of the lightguide.

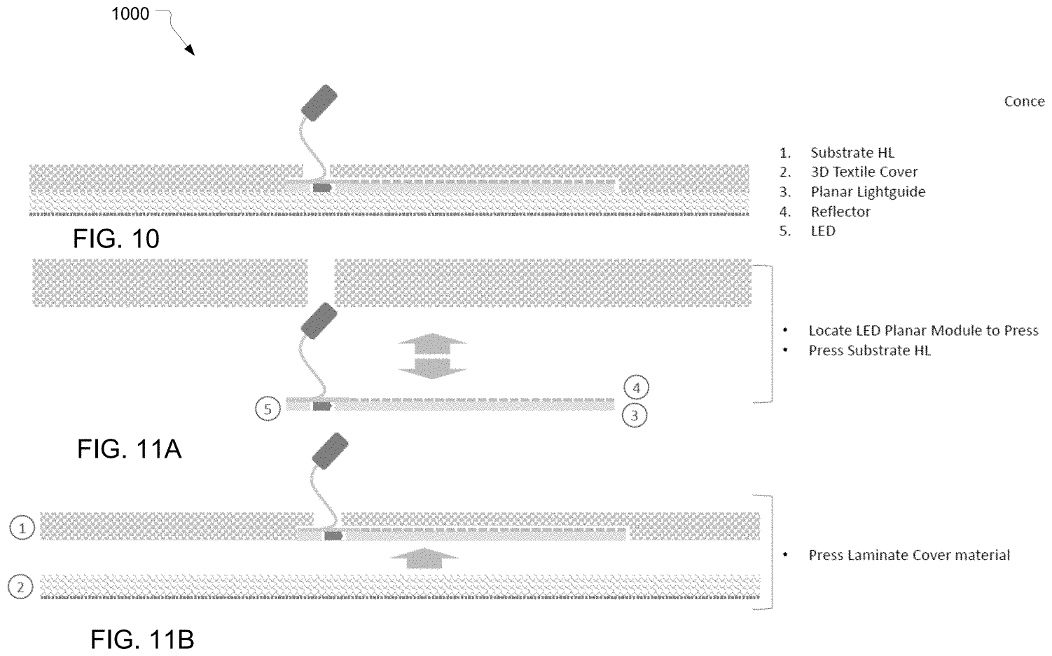

FIG. 10 is a cross-sectional view of another embodiment 1000 of an interior trim assembly including a lighting system consistent with the present disclosure and FIGS. 11A-11B illustrate successive steps in assembling the embodiment 1000 shown in FIG. 10. The illustrated example embodiment includes a substrate, a 3D textile cover and a planar light guide. The ambient and or UVC LEDS on the substrate 106 are positioned in a cavity formed in the substrate and relative light guide as shown to emit light through a bottom surface of the lightguide. Light emitted from the LEDS in a direction toward the reflector is reflected from the reflector an emitted from a bottom surface of the lightguide.

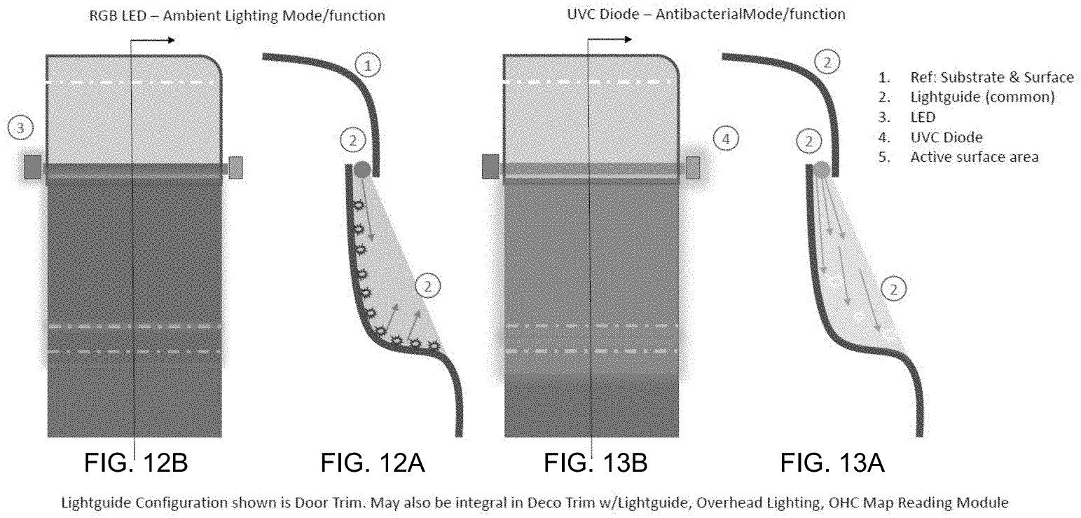

FIG. 12A is a side sectional diagrammatic view of a system consistent with the present disclosure positioned in a door trim assembly illuminating portions of a vehicle door with (as shown LED (RED) ON) and ambient light. FIG. 12B is a front diagrammatic view of the system shown in FIG. 12A showing portions of the door illuminated by the ambient light. FIG. 13A is a side sectional diagrammatic view of the system shown in FIG. 12A illuminating portions of a vehicle door with UVC light (as shown UVC Diode (Blue) ON). FIG. 13B is a front diagrammatic view of the system shown in FIG. 12A showing portions of the door illuminated by the UVC light.

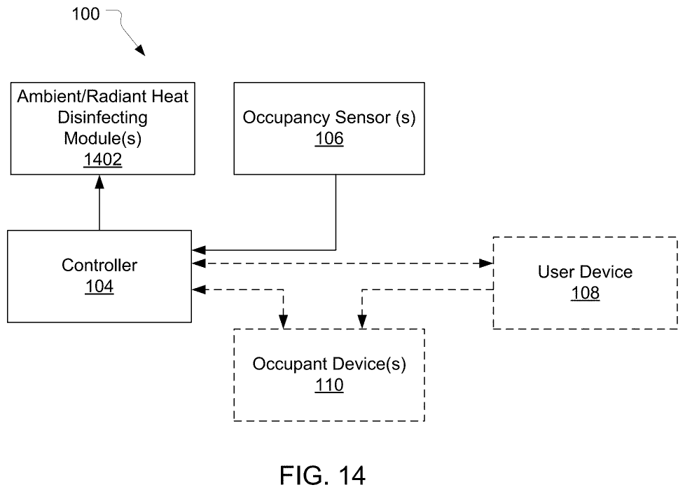

Turning now to FIG. 14, there is illustrated a simplified block diagram of another embodiment 1300 of a system consistent with the present disclosure. The illustrated example embodiment, includes one or more ambient or radiant heat disinfecting module(s) 1402 within a vehicle interior, a controller 104 configured to control operation of the modules 1402 to emit heat and one or more occupancy sensors 106. Reference to ambient heating is reference to the feature of heating up to about 40° C. Heating above 40° C. will then result a relatively higher and more noticeable amount of heat that may then radiate off of the surface of the selected location of the module in the vehicle.

The system may also include an optional user device 108 for providing user input to the controller 104 to activate and/or deactivate the modules 1402. The system may also or alternatively include one or more occupant devices 110 used by a present or future occupant of the vehicle interior, e.g. passengers. The controller 104, occupancy sensor(s) 106, user device(s) 108 and occupant device(s) 110 may be configured and may operate as described herein in connection with the system 100 described in connection with FIG. 1 and disinfection may be accomplished by the ambient heat disinfecting modules 1402.

The ambient heat disinfecting modules may be incorporated/integral and in certain cases embedded in a vehicle HVAC system and/or within vehicle trim and/or may be attached to the vehicle trim. To disinfect surfaces within the vehicle, the controller may provide control signals to the modules 1402 to cause the modules to heat the interior vehicle to a temperature and for a duration sufficient to deactivate viruses with in the vehicle. In some embodiments, for example, the modules 1402 may heat the interior of the vehicle to a temperature in a range from about 50° Celsius (C.) to 100° C. for a time period preferably in a range from about 1 minute to 20 minutes. Lower temperatures of the interior of the vehicle may then require relatively longer heating times. As described in connection with the system 100, control of the modules to provide disinfection with ambient heat may be incorporated into a ride-share application. Also, occupancy sensors may be incorporated into the modules 1402.

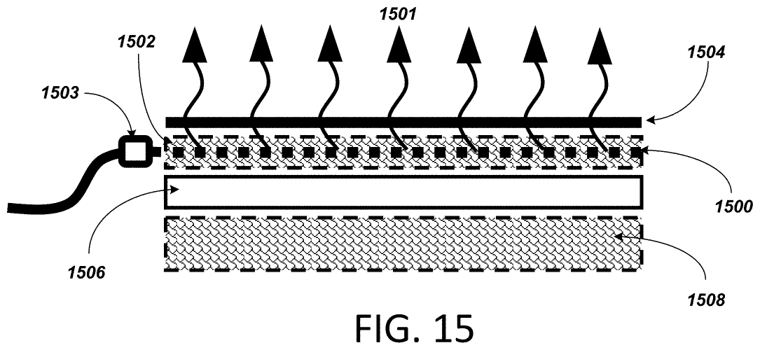

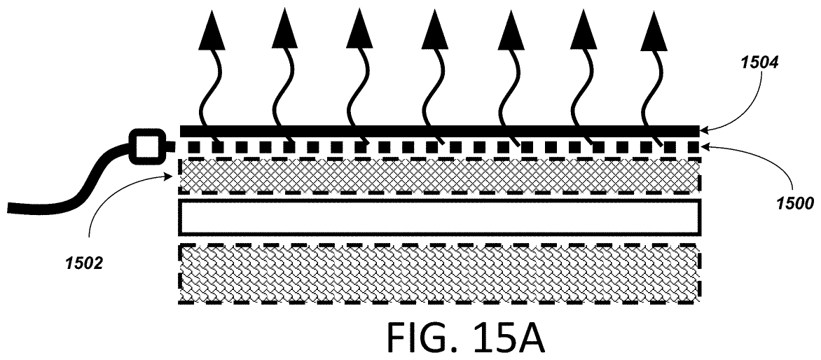

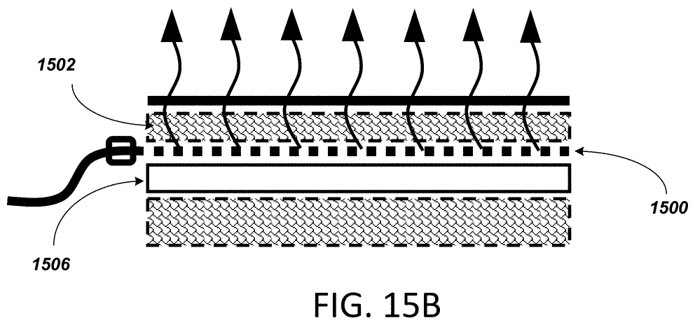

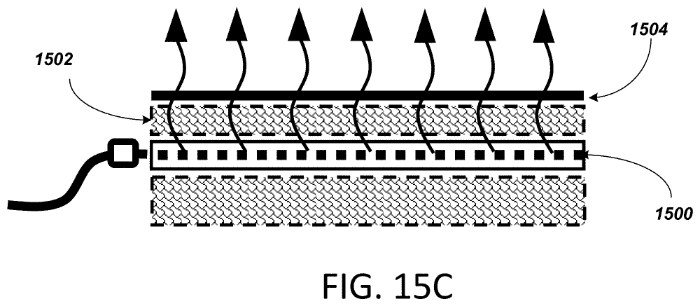

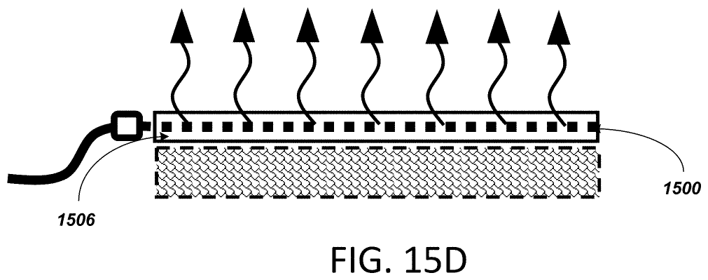

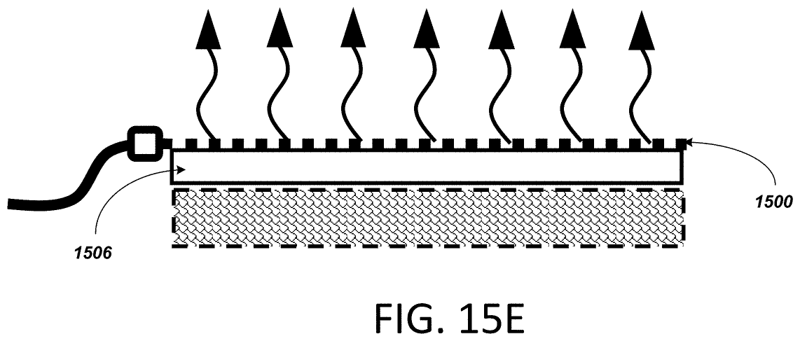

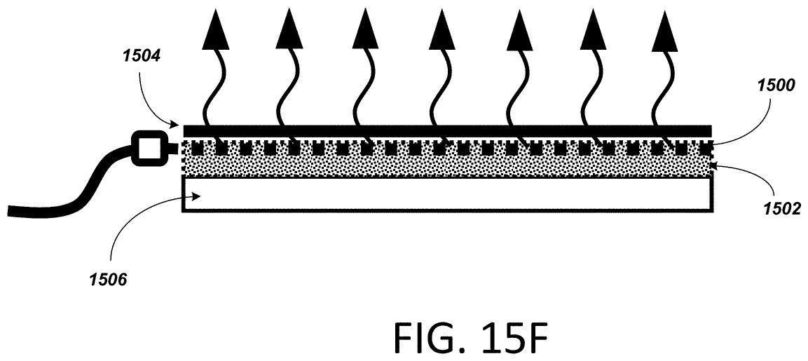

FIG. 15 illustrates one potential embodiment showing in cross-section of a trim panel the use of a plurality of heating elements 1500 providing heat flow 1501 within backing material 1502 with cover material 1504, substrate 1506 and thermal-insulation layer 1508. A temperature sensor is shown at 1503. FIG. 15A is similar to FIG. 15 except that the heating elements 1500 are positioned between the cover layer 1504 and backing material 1502. FIG. 15B illustrates the embodiment wherein the heating elements 1500 are laminated to the substrate 1506. FIG. 15C illustrates that situation where the heating elements 1500 are placed within the substrate 1506 under the backing material 1502 and cover layer. FIG. 15D illustrates placement of the heating elements 1500 within the substrate 1506 where there is no backing material or cover layer. FIG. 15E illustrates the heating elements 1500 on the surface of the substrate 1506. FIG. 15F illustrates heating elements 1500 within the backing layer 1502 which is beneath cover layer 1504.

Heating element 1500 may include wire, conductive fibers, conductive inks (e.g. carbon filled) or paste positioned within any trim panel material such as carbon nanotube paste, graphite, graphene or conductive polymeric or polymer resins where electrically conductive fillers are dispersed. Resins may include acrylonitrile-butadiene-styrene (ABS), polypropylene, and polycarbonate-ABS blends.

Backing materials 1502 may include foam material, such as polyurethane foam, polyolefin foam, or polysilioxane foam. The backing materials may also include non-woven material such made from PET. The backing materials may have a thickness in the range of 1.0 mm to 25.0 mm. Cover materials may be selected from polyvinyl chloride (PVC), thermoplastic olefins (TPO), thermoplastic elastomers (e.g. styrene-ethylene-butadiene block copolymers), leather, PVC/TPO blends. Cover materials may also be selected from textile material, such as polyester/polyurethane based material sold under the name ALCANTARA™. All such cover materials may have a thickness in the range of 0.5 mm to 10.0 mm, more preferably, 0.5 mm to 5.0 mm.

The substrate 1506 may be preferably selected from ABS, polypropylene (PP), PC-ABS. It may preferably have a thickness in the range of 0.5 mm to 15.0 mm. The level of thermally-conductive filler present in the substrate material may fall in the range of 1.0% (wt.) to 25.0% (wt.). The thermally-insulating layer 1506 may be selected from foam material, such as polyurethane foam. The thermally-insulating layer may have a thickness of 0.5 mm to 15.0 mm. The thermally-insulating layers is contemplated to direct the heat more efficiently to the surface of the cover materials or surface of the substrate and to reduce the heating cycle time for disinfection.

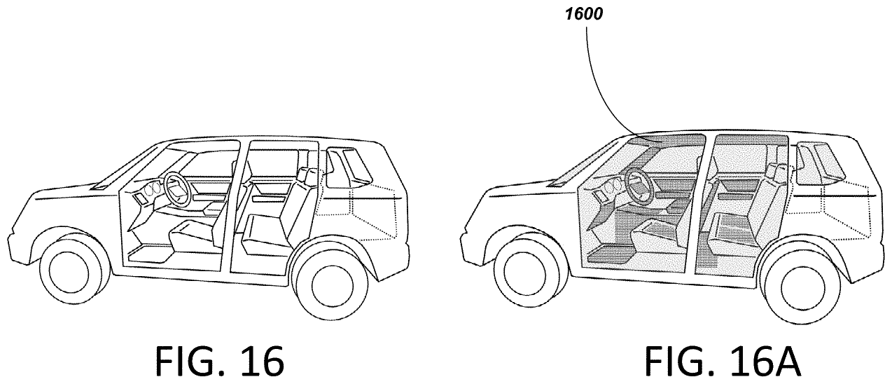

FIG. 16 shows a typical vehicle that may be configured to include the heating disinfecting modules and/or UVC disinfectant system herein. As can be seen in FIG. 16A, the shaded surfaces 1600 that may now be selectively heated and/or selectively exposed to UVC for disinfection include seating, headliners, instrumental panel portions, side panels, steering wheel, floor mats, overhead consoles, center consoles, armrests, glove-box container surfaces and/or sun visors. In such regards, the surfaces that can be configured to include the heating disinfecting modules or be exposed to UVC include those surfaces that are intended to be in contact with a given vehicle occupant or for which a vehicle occupant may typically engage.

Embodiments of the methods described herein may be implemented using a controller, processor and/or other programmable device. To that end, the methods described herein may be implemented on a tangible, non-transitory computer readable medium having instructions stored thereon that when executed by one or more processors perform the methods. The storage medium, e.g. memory, may include any type of tangible medium, for example, any type of disk optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of media suitable for storing electronic instructions.

The functions of the various elements shown in the figures, including any functional blocks labeled as “controller”, such as the controller 104, may be provided through the use of dedicated hardware as well as hardware capable of executing software in association with appropriate software. The functions may be provided by a single dedicated processor, by a single shared processor, or by a plurality of individual processors, some of which may be shared. Moreover, explicit use of the term “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

All definitions, as defined and used herein, should be understood to control over dictionary definitions, definitions in documents incorporated by reference, and/or ordinary meanings of the defined terms.

The term “coupled”, “interfaced”, “connected” or “communicating” as used herein refers to any connection, coupling, link or the like by which signals carried by one system element are imparted to the “coupled” element. Such “coupled” devices, or signals and devices, are not necessarily directly connected to one another and may be separated by intermediate components or devices that may manipulate or modify such signals. Likewise, the terms “connected” or “coupled” as used herein in regard to mechanical or physical connections or couplings is a relative term and does not require a direct physical connection. Elements, components, modules, and/or parts thereof that are described and/or otherwise portrayed through the figures to communicate with, be associated with, and/or be based on or in response to, something else, may be understood to so communicate, be associated with, and/or be based on in a direct and/or indirect manner, unless otherwise stipulated herein.

Throughout the entirety of the present disclosure, use of the articles “a” and/or “an” and/or “the” to modify a noun may be understood to be used for convenience and to include one, or more than one, of the modified noun, unless otherwise specifically stated. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Use of the phrase “in a range from X to Y” or “between X and Y” is meant to be inclusive of X and Y, unless otherwise specifically stated. The terms “comprising”, “including” and “having” are intended to be inclusive and mean that there may be additional elements other than the listed elements.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure. Future-filed applications claiming priority to this application may claim the disclosed subject matter in a different manner and generally may include any set of one or more limitations as variously disclosed or otherwise demonstrated herein.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A system for disinfecting surfaces in a space comprising:
a disinfecting module configured to disinfect surfaces in the space;
a user device;
a controller configured to provide a control signal to the disinfecting module;
an occupancy sensor configured to detect the presence of an occupant within the space;
said user device wirelessly coupled to said controller and configured to provide a user output to said controller to activate or deactivate the disinfecting module.

24. The system of claim 23 wherein said disinfecting module comprises a heating module configured to heat said surfaces and space for disinfection.

25. A system according to claim 24, wherein the heating module is configured to heat the surface and space to a temperature and for a duration sufficient to deactivate viruses on the surfaces.

26. A system according to claim 24, wherein the heating module is configured to heat the surface and space to a temperature in a range from about 50° Celsius (C.) to 100° C. for a time period in a range from about 1 minute to 20 minutes.

27. A system according to claim 24, wherein the space is a vehicle interior and the heating module is incorporated in a vehicle HVAC system and/or within vehicle trim.

28. A system according to claim 24, wherein the heating module comprises a plurality of heating elements positioned within a trim panel.

29. A system according to claim 23 wherein the user device comprises a ride-share application and wherein the user output is provided by the ride-share application.

30. A system according to claim 23 wherein the disinfecting module comprises a lighting module including at least one light source configured to emit UVC light having dominant wavelength between 200 nm and 280 nm.

31. A system according to claim 30, wherein the lighting module comprises at least one ambient lighting LED and the at least one light source on a common substrate.

32. A system according to claim 30, wherein the lighting module comprises the at least one light source and the occupancy sensor on a common substrate.

33. A system according to claim 30, wherein the controller is configured to provide a control signal to cause the at least one light source to emit the UVC light in response to an output of the occupancy sensor, whereby the UVC light is emitted when an output of the occupancy sensor indicates the space is unoccupied.

34. A system according to claim 23, wherein the occupancy sensor is disposed in the disinfecting module.

35. A system according to claim 23, further comprising an occupant device configured to receive a notification for notifying an occupant that disinfection of the space has been completed.

36. The system of claim 35 wherein the occupant device is wirelessly connected to the system and comprises a ride-share application.

37. A system according to claim 35, wherein the notification is provided to the occupant device from the user device or from the controller.

38. The system of claim 23 wherein said space is a vehicle interior and said user device receives confirmation that the vehicle interior is in process of being disinfected or has been disinfected.

39. The system of claim 23 wherein said space is a vehicle interior and wherein said disinfection module selectively applies disinfection to portions of said vehicle interior where occupants were located or anticipated to be located.
